(19) United States
(12) Patent Application Publication
(10) Pub. No.: US 2009/0053273 A1
(43) Pub. Date: Feb. 26, 2009

(54) Topical medication garment and system and method for providing topical medication

(76) Inventor: Adriana Le, Chicago, IL (US)

Correspondence Address: PATENTS+TMS, P.C., 2849 W. ARMITAGE AVE., CHICAGO, IL, 60647, US

(21) Appl. No.: 11/894,872

(22) Filed: Aug. 21, 2007

(51) Int. Cl. A61F 13/00; A61K 31/167; A61P 23/02

(52) U.S. Cl. 424/402; 514/626

(57) ABSTRACT

A topical medication garment, a system and a method provide topical medication. An impermeable garment may allow the topical medication to maintain contact with the skin of the user. The garment may prevent the topical medication from absorption into clothing or the undergarment itself. The entirety of the garment may be made from an impermeable material or a portion of the garment may be made from an impermeable material. An impermeable liner may be attached to and/or may be connected to an interior surface of the garment. The garment may have legging portions that reside on the upper thighs of the user when the garment is worn. The legging portions may secure the position of the garment on the user and providing coverage of topical medication applied to the crotch area and upper thighs. The garment may have a structure that resembles a t-shirt.

### BACKGROUND OF THE INVENTION

The present invention generally relates to a topical medication garment and a system and a method for providing topical medication. More specifically, the present invention relates to a topical medication garment and a system and a method for providing topical medication which may have an impermeable garment that allows the topical medication to maintain contact with the skin of the user. The garment may prevent the topical medication from absorption into clothing or the undergarment itself. The entirety of the garment may be made from an impermeable material or a portion of the garment may be made from an impermeable material. Alternatively, an impermeable liner may be attached to and/or may be connected to an interior surface of the garment. The garment may have legging portions that reside on the upper thighs of the user when the garment is worn. The legging portions may secure the position of the garment on the user and/or provide coverage of topical medication applied to the crotch area and/or upper thighs. The garment may have a structure that resembles a “t-shirt”.

The garment may provide an occlusive dressing for a cream, a gel, an ointment, a solution, a salve and/or other topical medication to a bikini area, a groin, genitalia, anus and/or upper thighs. The garment may have elastic bands at the waist and/or leg openings of the garment. The garment may be provided in different sizes to match the size of a waist of the user.

It is generally known to provide pre-treatment to improve the outcome and/or provide pain relief of such procedures as laser hair removal, hair waxing, piercing, tattooing, tweezing and shaving. Such pre-treatment may be provided by applying a topical agent, such as a topical cream, a gel, an ointment and/or a salve. Further, to enhance absorption of the topical agents, a non-occlusive dressing is applied. The non-occlusive dressing prevents absorption of the topical agent into the clothing, prevents other body parts from contacting the topical agents and avoids the disarray of the topical agents spreading to unintended areas. Applying a non-occlusive dressing to the bikini area, groin, genitalia, anus and upper thighs may be difficult, unwieldy and embarrassing.

Several common medical conditions require application of a topical agent, such as, for example, hemorrhoids, infection, dermatitis, itching, dryness and various types of rashes. Often the initial treatment involves application of a topical agent. Some topical agents may be used for prolonged periods of time, and some topical agents may require repeated applications. Topical agents used for common medical conditions may be difficult and unpleasant to handle.

For example, pre-treatment for laser hair removal traditionally involves application of a topical anesthetic cream, such as, for example, a lidocaine and/or prilocaine preparation, such as that provided in EMLA (Eutectic Mixture of Local Anesthetics) (trademark of AstraZaneca, Inc.). The topical anesthetic cream is applied to the treatment area approximately one to two hours before treatment. One-eighth of an inch thickness may be recommended for application of the topical anesthetic cream.

To maintain contact of the topical anesthetic cream with the skin, a non-occlusive dressing is worn over the topical medication to prevent absorption of the anesthetic cream into the clothing to prevent other body parts from contacting the anesthetic cream and to maintain moisture of the anesthetic cream. A polyvinylidene chloride (PVDC) film such as Saran Wrap (trademark of S.C. Johnson Home Storage, Inc.) is often used as a non-occlusive dressing over the topical anesthetic cream. Vinylide chloride may be polymerized with monomers, such as, for example, acrylic esters and unsaturated carboxyl groups, to form long chains of vinylide chloride. These chains of vinylide chloride may have tightly bound molecules' that prevent gas or water from moving through the PVDC film. The PVDC film may act as a barrier against oxygen, moisture and chemicals and may be resistant to oxygen, water, acids, bases, and solvents. Therefore, such film is often used to maintain contact of the topical anesthetic cream with the skin.

However, PVDC film may be difficult to position on the desired body part. Moreover, application of the PVDC film to secure the PVDC film to the body of the user may require the PVDC film to be wrapped around a body part and/or placed on body areas not treated with the topical anesthetic cream. Such application of the PVDC film often causes discomfort for the user. The difficulty of applying and wearing the PVDC film often causes the PVDC film to fall off of the body or move from the targeted skin areas. Movement of the PVDC film from the targeted skin areas may cause the topical medication to contact other body parts and/or clothing and may reduce the effectiveness of the topical medication.

A variety of absorbent articles are available. Absorbent undergarments, diapers and pads may be used for absorption of urine, feces, blood and discharge. However, absorbent articles are undesirable for use with topical medication because the topical medication may be absorbed by the garment and pulled away from the skin. Impermeable garments are also available and may be used for treatment of incontinence or administration of a medicinal agent.

For example, U.S. patent application Ser. No. 11/213,394 (hereinafter “the '394 application”) discloses a disposable non-woven undergarment that has a medicinal agent for treatment of various medical conditions that affect the lower torso. The undergarment may be utilized as post-treatment to laser hair removal by providing a soothing and cooling gel. The medicinal agent may be limited to the crotch region.

However, the undergarment of the '394 application has a “brief” structure of a close-fitting, legless undergarment that does not extend past the intersection of the crotch and the legs. The legless structure limits the coverage of the topical medication by the garment. Since the application of topical medication for laser hair removal extends past the intersection of the crotch and the legs to the uppermost section of the inner thigh, the undergarment does not aid in the administration of the topical medication to those regions of the body. Further, even if topical medication is not applied at the uppermost section of the inner thighs or the intersection of the crotch and the upper legs, the undergarment may not prevent movement of the topical medication. The topical medication may move from the crotch area to the uppermost section of the inner thigh where the skin is not covered by the undergarment.

Therefore, the undergarment of the '394 application does not provide the coverage needed for application of topical medication to the bikini area, the groin, the genitalia, the anus and/or the upper thighs, such as that needed for laser hair removal or waxing. Further, the leg openings of the “brief” style undergarment may shift position and ‘ride up’ on the user wearing the undergarment. Thus, the “brief” style undergarment limits the coverage of the topical medication by the undergarment. Moreover, placement of the leg openings of the “brief” style undergarment at the intersection of the crotch and the upper legs when the undergarment is worn may irritate the skin that is to be soothed, and the user may experience discomfort in the skin area to be treated.

A need, therefore, exists for a topical medication garment and a system and a method for providing topical medication. Additionally, a need exists for a topical medication garment and a system and a method for providing topical medication which may act as a dressing for topical medication applied to a bikini area, a groin, genitalia, an anus and/or upper thighs. Further, a need exists for a topical medication garment and a system and a method for providing topical medication which may prevent the topical medication from absorption into clothing or the undergarment itself. Still further, a need exists for a topical medication garment and a system and a method for providing topical medication which may restrict, may limit and/or may prevent movement of the topical medication to body parts to which the topical medication was not applied. Furthermore, a need exists for a topical medication garment and a system and a method for providing topical medication which may maintain the moisture of the topical medication. Still further, a need exists for a topical medication garment and a system and a method for providing topical medication which may restrict, may limit and/or may prevent unwanted movement of the topical medication to body parts to which the topical medication was not applied.

### SUMMARY OF THE INVENTION

The present invention generally relates to a topical medication garment and a system and a method for providing topical medication. More specifically, the present invention relates to a topical medication garment and a system and a method for providing topical medication which may have an impermeable garment that allows the topical medication to maintain contact with the skin of the user. The garment may prevent the topical medication from absorption into clothing or the undergarment itself. The entirety of the garment may be made from an impermeable material or a portion of the garment may be made from an impermeable material. Alternatively, an impermeable liner may be attached to and/or may be connected to an interior surface of the garment. The garment may have legging portions that reside on the upper thighs of the user when the garment is worn. The legging portions may secure the position of the garment on the user and/or provide coverage of topical medication applied to the crotch area and/or upper thighs. The garment may have a structure that resembles a “t-shirt”.

The garment may provide an occlusive dressing for a cream, a gel, an ointment, a solution, a salve and/or other topical medication to a bikini area, a groin, genitalia, anus and/or upper thighs. The garment may have elastic bands at the waist and/or leg openings of the garment. The garment may be provided in different sizes to match the size of a waist of the user.

To this end, in an embodiment of the present invention, a system for reducing irritation of skin of a user is provided. The system has a garment having an exterior surface and an interior surface wherein the interior surface of the garment is located in a position opposite to the exterior surface of the garment and further wherein the garment has a first layer of woven material and a second layer of impermeable non-woven material wherein the second layer of the impermeable material has an inner surface and an outer surface wherein the inner surface is located in a position opposite to the outer surface wherein the outer surface of the second layer of the impermeable non-woven material is connected to the first layer of the woven material. Further, the system has a first aperture in the garment wherein the aperture has a circumference; and a second aperture and a third aperture in the garment wherein the third aperture is located in a position opposite to the second aperture. Still further the system has a left limb section of the garment wherein the left limb section extends from the second aperture of the garment; and a right limb section of the garment wherein the right limb section extends from the third aperture of the garment. Moreover, the system has a topical medication affixed to the layer of impermeable non-woven material.

In an embodiment, the first layer of the impermeable non-woven material covers an entirety of the interior surface of the garment.

In an embodiment, the second layer of the woven material covers an entirety of the exterior surface of the garment.

In an embodiment, the system has a central panel of the garment wherein the second layer of the impermeable non-woven material only resides in the central panel.

In an embodiment, the system has an elastic band at the first aperture wherein the elastic band has a circumference equal to the circumference of the first aperture.

In an embodiment, the system has side panels of the garment wherein the first layer of the woven material only resides in the side panels.

In an embodiment, the system has a fourth aperture of the garment wherein the fourth aperture is located in a position opposite to the first aperture.

In another embodiment of the present invention, a garment to be worn by a user for treatment of skin of the user wherein the user has a waist, a left arm, a right arm, a left leg and a right leg is provided. The garment has a front portion and a second portion connected to the front portion. Further, the garment has a first aperture formed by the front portion and the back portion; a left limb portion connected to the front portion and the back portion wherein the left limb portion extends from the front portion and the back portion; and a right limb portion connected to the front portion and the back portion wherein the right limb portion extends from the front portion and the back portion. Still further, the garment has an interior surface and an exterior surface formed by the front portion, the back portion, the left limb portion and the right limb portion wherein the interior surface is located in a position opposite to the exterior surface. Moreover, the garment has a topical medication associated with the interior surface.

In an embodiment, the front portion, the back portion, the left portion and the right portion are manufactured from a non-woven impermeable material.

In an embodiment, the left limb portion is in a position opposite to the first aperture and the right limb portion is in a position opposite to the first aperture.

In an embodiment, the garment has a second aperture located in a position opposite to the first aperture wherein the left limb portion and the right limb portion are adjacent to the second aperture.

In an embodiment, the garment has a liner having the topical medication wherein the liner is connected to the front portion, the back portion, the left limb portion and the right limb portion.

In an embodiment, the front section is manufactured from a non-woven impermeable material.

In an embodiment, the garment has an inner layer attached to the front portion, the back portion, the left portion and the right portion wherein the inner layer is manufactured from an impermeable non-woven material.

In an embodiment, the garment has an outer layer attached to the front portion, the back portion, the left portion and the right portion wherein the outer layer is manufactured from a woven material.

In an embodiment, the front portion is integral with the back portion.

In an embodiment, the garment has lidocaine in the topical medication.

In another embodiment of the present invention, a method for pre-treating skin of a user with topical medication wherein the user has a crotch area, a waist, a left leg and a right leg is provided. The method has the steps of: providing a garment that has an impermeable non-woven material wherein the garment extends from the waist of the user to the crotch area of the user and onto the left leg and the right leg of the user wherein the garment is manufactured from the impermeable non-woven material; associating a topical medication with the impermeable non-woven material of the garment; and clothing the user in the garment wherein clothing the user in the garment transfers the topical medication to the skin of the user.

In an embodiment, the impermeable non-woven material only covers the crotch area of the user.

In an embodiment, the method has the step of incorporating lidocaine in the topical medication.

It is, therefore, an advantage of the present invention to provide a topical medication garment and a system and a method for providing topical medication.

Another advantage of the present invention is to provide a topical medication garment and a system and a method for providing topical medication which may act as a dressing for topical medication applied to a bikini area, a groin, genitalia, an anus and/or upper thighs.

Yet another advantage of the present invention is to provide a topical medication garment and a system and a method for providing topical medication which may prevent the topical medication from absorption into clothing or the undergarment itself.

A still further advantage of the present invention is to provide a topical medication garment and a system and a method for providing topical medication which may restrict, may limit and/or may prevent movement of the topical medication to body parts to which the topical medication was not applied.

Another advantage of the present invention is to provide a topical medication garment and a system and a method for providing topical medication which may maintain the moisture of the topical medication.

Yet another advantage of the present invention is to provide a topical medication garment and a system and a method for providing topical medication which may provide a garment having a central panel of impermeable material.

A still further advantage of the present invention is to provide a topical medication garment and a system and a method for providing topical medication which may have an elastic waistband and/or elastic leg openings.

Another advantage of the present invention is to provide a topical medication garment and a system and a method for providing topical medication which may have side panels made from a woven material.

And, another advantage of the present invention is to provide a topical medication garment and a system and a method for providing topical medication which may be made entirely from an impermeable material.

Still further, an advantage of the present invention is to provide a topical medication garment and a system and a method for providing topical medication which may provide a garment having an inner lining made from an impermeable material.

Yet another advantage of the present invention is to provide a topical medication garment and a system and a method for providing topical medication which may prevent the need for repeated applications of the topical medication.

A still further advantage of the present invention is to provide a topical medication garment and a system and a method for providing topical medication which may enable the user to perform various activities while wearing the garment.

Another advantage of the present invention is to provide a topical medication garment and a system and a method for providing topical medication which may secure the position of the garment on the user.

A still further advantage of the present invention is to provide a topical medication garment and a system and a method for providing topical medication which may have leg openings of the garment placed away from the crotch area of the user and, thus, prevent irritation of skin at the crotch area of the user.

And, another advantage of the present invention is to provide a topical medication garment and a system and a method for providing topical medication which may decrease irritation of the skin of the user adjacent to the garment.

Moreover, an advantage of the present invention is to provide a topical medication garment and a system and a method for providing topical medication which may provide a seamless undergarment.

Additional features and advantages of the present invention are described in, and will be apparent from, the detailed description of the presently preferred embodiments and from the drawings.

### BRIEF DESCRIPTION OF THE DRAWINGS

FIG. 1 illustrates a perspective view of a garment in an embodiment of the present invention.

FIG. 2A illustrates a front view of a garment in an embodiment of the present invention.

FIG. 2B illustrates a cross-sectional view taken generally along line IIA-IIA of FIG. 2A of a garment in an embodiment of the present invention.

FIG. 3 illustrates a cross-sectional view taken generally along line IIA-IIA of FIG. 2A of a garment in an embodiment of the present invention.

FIG. 4 illustrates a front view of a garment in an embodiment of the present invention.

FIG. 5 illustrates a perspective view of a garment in an embodiment of the present invention.

FIG. 6 illustrates a cross-sectional view taken generally along line IIA-IIA of FIG. 2A of a garment in an embodiment of the present invention.

FIG. 7 illustrates a perspective view of a garment in an embodiment of the present invention.

FIG. 8A illustrates a front view of a garment in an embodiment of the present invention.

FIG. 8B illustrates a cross-sectional view taken generally along line VIIIA-VIIIA of FIG. 8A of a garment in an embodiment of the present invention.

### DETAILED DESCRIPTION OF THE PRESENTLY PREFERRED EMBODIMENTS

The present invention generally relates to a topical medication garment and a system and a method for providing topical medication. More specifically, the present invention relates to a topical medication garment and a system and a method for providing topical medication which may have an impermeable garment that allows the topical medication to maintain contact with the skin of the user. The garment may prevent the topical medication from absorption into clothing or the undergarment itself. The entirety of the garment may be made from an impermeable material or a portion of the garment may be made from an impermeable material. Alternatively, an impermeable liner may be attached to and/or may be connected to an interior surface of the garment. The garment may have legging portions that reside on the upper thighs of the user when the garment is worn. The legging portions may secure the position of the garment on the user and/or provide coverage of topical medication applied to the crotch area and/or upper thighs. The garment may have a structure that resembles a “t-shirt”.

The garment may provide an occlusive dressing for a cream, a gel, an ointment, a solution, a salve and/or other topical medication to a bikini area, a groin, genitalia, anus and/or upper thighs. The garment may have elastic bands at the waist and/or leg openings of the garment. The garment may be provided in different sizes to match and/or to fit the size of a user. A size of the elastic band at the waist of the garment may be based on and/or may be relative to a circumference of a waist of the user.

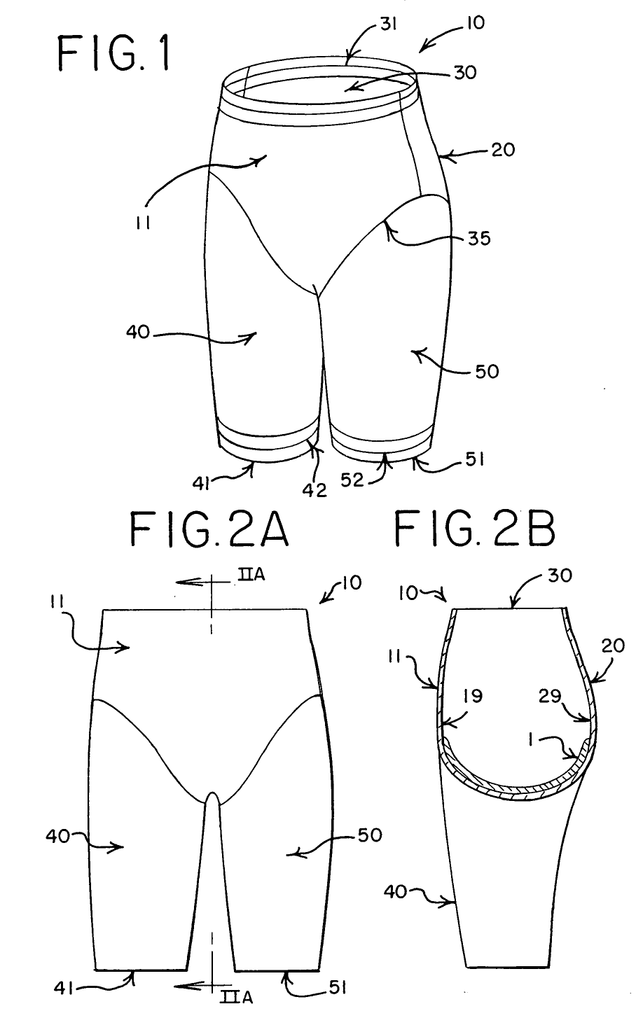

Referring now to the drawings wherein like numerals refer to like parts, FIGS. 1, 2A and 2B illustrate a garment 10 in an embodiment of the present invention. The garment 10 may be worn, for example, as an undergarment or otherwise secured around the waist of an individual that has a topical medication 1 applied to a bikini area, a groin, genitalia, anus and/or upper thighs. A front portion 11 of the garment 10 may be integral with, connected to and/or attached to a back portion 20 of the garment 10. The front portion 11 and the back portion 20 may form a “brief”-style undergarment structure. For example, the front portion 11 may be connected to the back portion 20 by stitching. The front portion 11 may reversibly attach to the back portion 20 using, for example, fasteners, zippers, ties, slide, buttons, hook and loop fasteners, Velcro (trademark of Velcro Industries) and/or the like, which may secure the garment 10 around the user. Fasteners may allow the front portion 11 to attach to the back portion 20 and/or allow the front portion 11 to separate from the back portion 20. In a preferred embodiment, the front portion 11 and the back portion 20 may be integrally formed with one another.

The front portion 11 and the back portion 20 may form a waist opening 30 of the garment 10. The waist opening 30 may be sized to match and/or to fit the size of a waist of the user. The garment 10 may have a left legging 40 and/or a right legging 50. The left legging 40 and/or the right legging 50 may extend from the front portion 11 and/or the back portion 20 of the garment 10. The left legging 40 and/or the right legging 50 may be integral with, connected to and/or attached to the front portion 11 and/or the back portion 20. In a preferred embodiment, the left legging 40 and/or the right legging 50 may be integral with the front portion 11 and the back portion 20. If the garment is worn by a user, the left legging 40 and/or the right legging 50 may reside on the left leg and the right leg of the user, respectively, and may extend to the thighs of the user. The left legging 40 and/or the right legging 50 may have a left opening 41 and/or a right opening 51, respectively. As a result, the left leg of the user may pass through the left opening 41 and the right leg of the user may pass through the right opening 51. The left legging 40 and/or the right legging 50 may increase an area of skin covered by the garment 10 relative to a “brief”-style undergarment.

The front portion 11, the back portion 20, the left legging 40 and the right legging 50 may be manufactured from a non-woven impermeable material, such as, for example, polyacrylates, polyvinyl acetates, wax, polyethylene, a synthetic fiber such as neoprene, Spandex (trademark of Monsanto Chemical Company) and/or elastane, and/or the like. It should be understood that the non-woven impermeable material may be any material that does not absorb the topical medication 1 as known to one having ordinary skill in the art.

As generally shown in FIG. 2B, the front portion 11 may have a front interior surface 19 and/or the back portion 20 may have a back interior surface 29. The front interior surface 19 and/or the back interior surface 29 may cover the areas of skin that have the topical medication 1 applied thereon. The topical medication 1, may be, for example, lidocaine, prilocaine and/or benzocaine. In a preferred embodiment the topical medication 1 is an emulsion preparation having lidocaine and/or prilocaine. The topical medication 1 should not be limited to a specific embodiment of the topical medication 1. It should be understood that the topical medication 1 may be any topical medication that may be applied to the skin of the user as known to one having ordinary skill in the art.

The topical medication 1 may be associated with and/or affixed to the garment 10. For example, the topical medication 1 may be incorporated into the material of the garment 10 during manufacture of the garment 10. Alternatively, the topical medication 1 may be associated with the garment 10 by a laminate layer. The topical medication 1 may have a chemical compound that adheres the topical medication 1 to the garment and allows the topical medication 1 to transfer to the skin of the user. The topical medication 1 may be formulated so that the topical medication 1 may be associated with the garment 10 at room temperature, for example, as a gel, but may liquify or otherwise transform if body heat is exerted on, transferred to or otherwise subjected to the topical medication 1. For example, body heat due to the wearing of the garment 10 by the user may liquify the topical medication 1 and transfer the topical medication 1 to the skin of the user. Thus, wearing the garment 10 may transfer the topical medication 1 to the targeted areas of the skin.

If the topical medication 1 is to be applied to a frontal area of the user, the front portion 11 may be manufactured from the non-woven impermeable material having the topical medication 1, and the back portion 20 may be manufactured from a woven material. If the topical medication 1 is to be applied to a rear area of the user, the back portion 20 may be manufactured from the non-woven impermeable material having the topical medication 1, and the front portion 11 may be manufactured from a woven material. The woven material may be, for example, a fabric, a cotton, a lace, a stretchable fabric, a wicking fabric, an absorbant fabric and/or the like. In a preferred embodiment, the woven material may absorb and/or may wick sweat, water, moisture and/or the like. It should be understood that the woven material may be any material as known to one having ordinary skill in the art.

As generally shown in FIG. 1, the garment may have a first elastic band 31 at the waist opening 30, a second elastic band 42 at the left leg opening 41 and/or a third elastic band 52 at the right leg opening 51. The first elastic band 31 may be sized to match the size of a waist of the user. The first elastic band 31, the second elastic band 42 and/or the third elastic band 52 may tighten the garment 10 on the user. Due to the first elastic band 31, the waist opening 30 may match the size of the waist of the user. Due to the second elastic band 42 and/or the third elastic band 52, the left leg opening 41 and/or the right leg opening 41 may match a size of the left arm of the user and/or a size of the right arm of the user, respectively. Therefore, the topical medication 1 may be prevented from moving from areas of skin where the topical medication 1 is applied to areas of skin not covered by the garment 10. The first elastic band 31, the second elastic band 42 and/or the third elastic band 52 may prevent movement of the garment 10. Therefore, the garment 10 may maintain a position relative to the user.

The garment 10 may be provided in different sizes to match the size of a user. A size of the first elastic band 31 at the waist opening 30 of the garment 10 may be based on and/or may be relative to a circumference of a waist of the user. Due to the elastic bands 31, 42, 52 holding the garment 10 in place on the user, the garment 10 may be loose fitting for the user. Therefore, the garment 10 may provide comfort for the user and may prevent the topical medication 1 applied to the user from contacting the clothing and/or other areas of the body of the user. The left legging 40, the right legging 50 and/or the elastic bands 31, 42, 52 may prevent the need for contact of sections 35 of the garment 10 at the intersection of the crotch and the legs of the user with the skin of the user. Therefore, the garment 10 may prevent irritation of the skin at which the topical medication 1 is likely to be applied by transfer of the topical medication 1 from the garment 10.

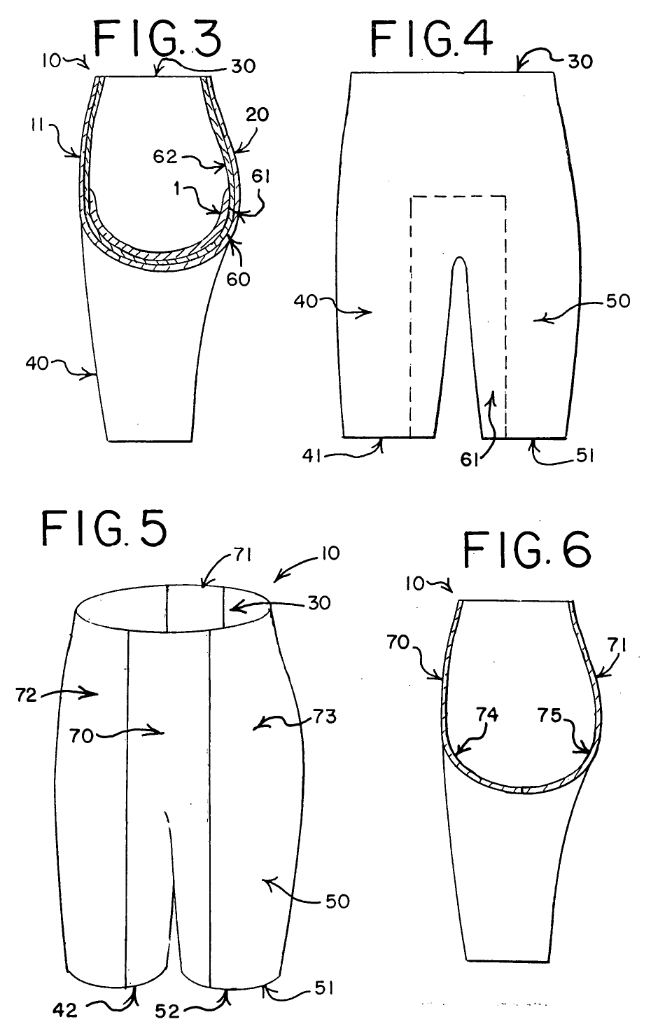

As generally shown in FIG. 3, the garment 10 may have two layers. The garment 10 may have an outer layer 60 of the woven material and/or an inner layer 61 of the non-woven impermeable material. The inner layer 61 may be attached to, affixed to, connected to and/or integrally formed with the outer layer 60. The non-woven impermeable inner layer 61 may be attached to the woven outer layer 60 by stitching that may have loops of thread, for example, through the non-woven impermeable inner layer 61 and the woven outer layer 60. Alternatively, the non-woven impermeable inner layer 61 may be attached to the woven outer layer 60 by an adhesive and/or the like. In an embodiment generally shown in FIG. 4, the non-impermeable inner layer 61 may be located at the crotch region and/or the inner thighs of the user. The location of the non-woven impermeable inner layer 61 may be selected based on a location that the topical medication 1 is applied. Alternatively, the entirety of the garment 10 may be lined with the non-woven impermeable inner layer 61. The inner layer 61 may have an inner interior surface 62. The inner interior surface 62 may cover the areas of skin that have the topical medication 1 applied thereon. The topical medication 1 may be applied to the inner interior surface 62 so that wearing the garment 10 transfers the topical medication 1 to the skin of the user.

As generally shown in FIGS. 5 and 6, a central front panel 70 and/or a central back panel 71 may be made of the impermeable non-woven material. A left side panel 72 and/or a right side panel 73 may be made of the woven material. The left side panel 72 and/or the right side panel 73 may be connected to the central front panel 70 and/or a central back panel 71, such, as for example, by stitching of loops of thread through the central front panel 70, the central back panel 71, the left side panel 72 and/or the right side panel 73. The central front panel 70 may have a front interior surface 74, and/or the central back panel 71 may have a back interior surface 75. The front interior surface 74 and/or the back interior surface 75 may cover the areas of skin that may have the topical medication 1 applied thereon. The topical medication 1 may be applied to the front interior surface 74 and/or the back interior surface 75 so that wearing the garment 10 may transfer the topical medication 1 to the skin of the user. The left side panel 72 and/or the right side panel 73 may absorb moisture, water and/or sweat from the user.

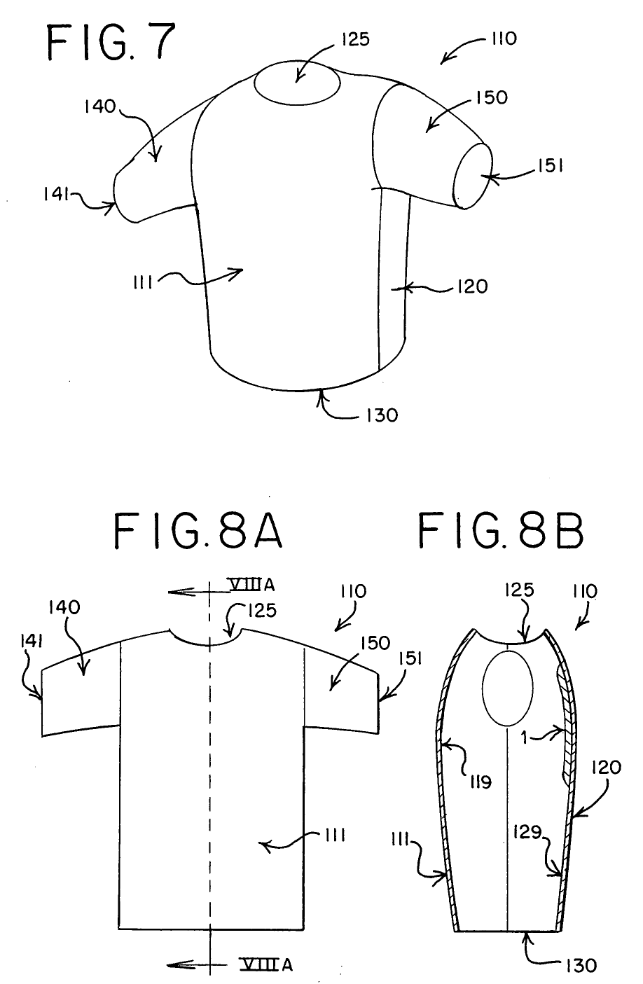

FIGS. 7, 8A and 8B generally illustrate another embodiment of a garment 110 of the present invention. The garment 110 may be worn, for example, as an undergarment or otherwise worn on an upper torso of the user. The garment 110 may be used for pre-treatment for laser hair removal. The pre-treatment for laser hair removal may have application of the topical medication 1 to a back, shoulders, a chest and/or a stomach. Laser removal of unwanted hair may be a transfer of laser energy to a hair follicle and/or other like treatment of the skin of the user. A front portion 111 of the garment 110 may be integral with, connected to and/or attached to a back portion 120 of the garment 110. The front portion 111 and the back portion 120 may form a “t-shirt”-style undergarment structure. A “t-shirt”-style undergarment structure may be a pullover shirt that is close-fitting and with a round neckline and short sleeves. For example, the front portion 111 may be connected to the back portion 120 by stitching that may have loops of thread, for example, through the front portion 111 and the back portion 120. The front portion 111 may reversibly attach to the back portion 120 using, for example, fasteners, zippers, ties, slide, buttons, hook and loop fasteners, Velcro (trademark of Velcro Industries) and/or the like, which may secure the garment 110 around the user. Fasteners may allow the front portion 111 to attach to the back portion 120 and/or allow the front portion 111 to separate from the back portion 120. In a preferred embodiment, the front portion 111 and the back portion 120 may be integrally formed with one another.

The front portion 111 and the back portion 120 may form a head opening 125 and/or a waist opening 130 of the garment 110. The waist opening 130 may be sized to match and/or to fit the size of a waist of the user. The garment 110 may have a left arm 140 and/or a right arm 150. The left arm 140 and/or the right arm 150 may extend from the front portion 111 and/or the back portion 120 of the garment 110. The left arm 140 and/or the right arm 150 may be integral with, connected to and/or attached to the front portion 111 and/or the back portion 120. In an embodiment, the left arm 140 and/or the right arm 150 may be integral with the front portion 111 and the back portion 120. If the garment 110 is worn by the user, the left arm 140 and/or the right arm 150 may reside on the left arm and right arm of the user, respectively. The left arm 140 and/or the right arm 150 may have a left arm opening 141 and/or a right arm opening 151, respectively. As a result, the left arm of the user may pass through the left arm opening 141, and the right arm of the user may pass through the right arm opening 151.

The front portion 111, the back portion 120, the left arm 140 and the right arm 150 may be manufactured from non-woven impermeable material, such as, for example, polyacrylates, polyvinyl acetates, wax, polyethylene, a synthetic fiber such as neoprene, Spandex (trademark of Monsanto Chemical Company) and/or elastane, and/or the like. It should be understood that the non-woven impermeable material may be any material that does not absorb the topical medication 1 as known to one having ordinary skill in the art.

As generally shown in FIG. 8B, the front portion 111 may have a front interior surface 119, and/or the back portion 120 may have a back interior surface 129. The front interior surface 119 and/or the back interior surface 129 may cover the areas of skin that have the topical medication 1 applied thereon. The topical medication 1, may be, for example, lidocaine, prilocaine and/or benzocaine. In an embodiment, the topical medication 1 is a 5% emulsion preparation having lidocaine and prilocaine. The topical medication 1 should not be limited to a specific embodiment of the topical medication 1. It should be understood that the topical medication 1 may be any topical medication 1 that may be applies to the skin of the user as known to one having ordinary skill in the art.

The topical medication 1 may be associated with and/or affixed to the garment 110. For example, the topical medication 1 may be incorporated into the material of the garment 110 during manufacture of the garment 110. Alternatively, the topical medication 1 may be associated with the garment 110 by a laminate layer. The topical medication 1 may have a chemical compound that adheres the topical medication 110 to the garment and allows the topical medication 1 to transfer to the skin of the user. The topical medication 1 may be formulated so that the topical medication 1 may be associated with the garment 110 at room temperature, for example, as a gel, but may liquify or otherwise transform if body heat is exerted on, transferred to or otherwise subjected to the topical medication 1. For example, body heat due to the wearing of the garment 110 by the user may liquify the topical medication 1 and transfer the topical medication 1 to the skin of the user. Thus, wearing the garment 10 may transfer the topical medication 1 to the targeted areas of the skin.

If the topical medication 1 is to be applied to a frontal area of the user, the front portion 111 may be manufactured from the non-woven impermeable material having the topical medication 1, and the back portion 120 may be manufactured from a woven material. If the topical medication 1 is to be applied to a rear area of the user, the back portion 120 may be manufactured from the non-woven impermeable material having the topical medication 1, and the front portion 111 may be manufactured from a woven material. The woven material may be, for example, a fabric, a cotton, a lace, a stretchable fabric, a wicking fabric, an absorbant fabric and/or the like. In a preferred embodiment, the woven material may absorb and/or may wick sweat, water, moisture and/or the like. It should be understood that the woven material may be any material as known to one having ordinary skill in the art.

It should be understood that various changes and modifications to the presently preferred embodiments described herein will be apparent to those skilled in the art. Such changes and modifications may be made without departing from the spirit and scope of the present invention and without diminishing its attendant advantages. It is, therefore, intended that such changes and modifications be covered by the appended claims.

1. A system for reducing irritation of skin of a user, the system comprising:
a garment having a central panel, a left side panel and a right side panel wherein the central panel has a front portion and a back portion wherein the front portion of the central panel is located in a position opposite to the back portion of the central panel and further wherein the left side panel is located in a position opposite to the right side panel wherein the left side panel is connected to the front portion of the central panel and the back portion of the central panel wherein the right side panel is connected to the front portion of the central panel and the back portion of the central panel wherein the central panel has an exterior surface and an interior surface wherein the interior surface of the central panel is located in a position opposite to the exterior surface of the central panel and further wherein the central panel is manufactured from an impermeable nonwoven material;
a first aperture in the garment wherein the aperture has a circumference wherein the first aperture is located between the front portion of the central panel and the back portion of the central panel;
a second aperture and a third aperture in the garment wherein the third aperture is located in a position opposite to the second aperture;
a left limb section of the garment wherein the left limb section extends from the second aperture of the garment;
a right limb section of the garment wherein the right limb section extends from the third aperture of the garment; and
a topical medication affixed to the interior surface of the central panel.

2. The system of claim 1 wherein the left side panel and the right side panel are manufactured from the impermeable nonwoven material.

3. The system of claim 1 wherein the left side panel and the right side panel are manufactured from a woven material.

4. The system of claim 1 wherein the left limb section is formed by the central panel and the left side panel and further wherein the right limb section is formed by the central panel and the right side panel.

5. The system of claim 1 wherein the left limb section is formed by only the left side panel and further wherein the right limb section is formed by only the right side panel.

6. The system of claim 1 further comprising:
an emulsion of prilocaine and lidocaine located in the topical medication.

7. The system of claim 1 further comprising:
a fourth aperture of the garment wherein the fourth aperture is located in a position opposite to the first aperture and further wherein the fourth aperture is located between the front portion of the central panel and the back portion of the central panel.

8. A garment to be worn by a user for treatment of skin of the user wherein the user has a waist, a left arm, a right arm, a left leg and a right leg, the garment comprising:
a front portion;
a back portion connected to the front portion;
a first aperture formed by the front portion and the back portion;
a left limb portion connected to the front portion and the back portion wherein the left limb portion extends from the front portion and the back portion wherein the left limb portion has a left inner thigh section;
a right limb portion connected to the front portion and the back portion wherein the right limb portion extends from the front portion and the back portion wherein the right limb portion has a right inner thigh section adjacent to the left limb portion wherein the left inner thigh section is adjacent to the right limb portion;
an interior surface and an exterior surface formed by the front portion, and the back portion, the left limb portion and the right limb portion wherein the interior surface is located in a position opposite to the exterior surface;
a liner of impermeable nonwoven material connected to the interior surface wherein the liner is connected to the front portion, the left inner thigh section and the right inner thigh section; and
a topical medication associated with the liner.

9. The garment of claim 8 wherein the front portion, the back portion, the left limb portion and the right limb portion are manufactured from a woven material.

10. The garment of claim 8 wherein the left limb portion is in a position opposite to the first aperture and the right limb portion is in a position opposite to the first aperture.

11. The garment of claim 8 further comprising:
a second aperture located in a position opposite to the fist aperture wherein the left limb portion and the right limb portion are adjacent to the second aperture.

12. The garment of claim 8 wherein the left limb portion has a length defined by a top end and a bottom end wherein the top end is adjacent to the front portion of the garment and the back portion of the garment and further wherein the liner of impermeable non-woven material extends to the bottom end of the left limb portion.

13. The garment of claim 8 wherein the front section has a length defined by a top end and a bottom end wherein the bottom end is adjacent to the left limb portion and the right limb portion and further wherein the liner does not extend to the top end of the front section.

14. The garment of claim 8 further comprising:
an elastic band located at the first aperture wherein the first aperture has a circumference and further wherein the elastic band has a circumference equal to the circumference of the first aperture.

15. The garment of claim 8 further comprising:
an elastic band connected to the left limb portion wherein the left limb portion has a length defined by a top end and a bottom end wherein the top end is adjacent to the front portion of the garment and the back portion of the garment and further wherein the elastic band is connected to the bottom end of the left limb portion.

16. The garment of claim 8 wherein the front portion is reversibly attached to the back portion wherein the front portion may detach and then re-attach to the back portion.

17. The garment of claim 8 further comprising:
an emulsion of prilocaine and lidocaine located in the topical medication.

18. A method for pre-treating skin of a user with topical medication wherein the user has a crotch area, a waist, a left inner thigh, a right inner thigh, a left leg and a right leg, the method comprising the steps of:
providing a garment that has an impermeable non-woven material wherein the garment extends from the waist of the user to the crotch area of the user and onto the left leg and the right leg of the user wherein the impermeable non-woven material only covers the crotch area, the left inner thigh and the right inner thigh of the user; associating a topical medication with the impermeable non-woven material of the garment; and
clothing the user in the garment wherein clothing the user in the garment transfers the topical medication to the skin of the user.

19. The method of claim 18 wherein the impermeable non-woven material is connected to an outer layer of woven material.

20. The method of claim 18 wherein the topical medication has an emulsion of lidocaine and prilocaine.
